(19) United States
(12) Patent Application Publication
(10) Pub. No.: US 2010/0086499 A1
(43) Pub. Date: Apr. 8, 2010

(54) TOOTH WHITENING COMPOSITION

(76) Inventors: Gary Robert Burnett, Surrey (GB); Jennifer Jane Gordon, Surrey (GB); Jose Luis Velada, Surrey (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/300,351

(22) PCT Filed: May 11, 2007
(86) PCT No.: PCT/EP07/54601

§ 371 Date: Apr. 3, 2009

(30) Foreign Application Priority Data
May 9, 2006 (AU) PV 2006902434

(51) Int. Cl. A61K 8/20; A61Q 11/00

(52) U.S. Cl. 424/53

(57) ABSTRACT

The present invention provides an effective tooth whitening composition comprising a chlorine dioxide precursor, an organic acid anhydride and an orally acceptable carrier.

The present invention relates to a tooth whitening composition for bleaching tooth enamel. Specifically the present invention relates to a tooth whitening composition comprising chlorine dioxide.

White teeth have long been considered cosmetically desirable. Unfortunately, teeth almost invariably become discoloured in the absence of intervention. The tooth structure which is generally responsible for presenting a stained appearance is the enamel layer. Several factors contribute to enamel discoloration, but the three main factors are believed to be: (i) formation of plaque and tartar matrices on the tooth surface which then entrap stains; (ii) ingestion of certain drugs during gestational tooth formation; and (iii) discoloration due to oral cavity traumatization following or during which blood break-down products seep into the mineralized area of the teeth during enamel formation. This invention is primarily concerned with the first factor of tooth discoloration, that is, the natural stain which accumulates on teeth due to the formation of plaque and tartar.

Over-the-counter tooth whitening preparations have been developed to address the cosmetic preference of many to restore luster to tooth enamel discolored by surface entrapped materials. While all dentifrices and mouthwashes contain some cleaning and polishing agents, some enamel deposits become intractable to being fully removed by these agents under normal use conditions. For example, smokers often develop discolored enamel because the tars and particulates in exhaled cigarette smoke collect on the teeth. Further, a number of comestibles, such as tea, or some medicinal agents, can stain or discolor tooth enamel.

There are various approaches to enamel whitening currently in general use. One approach is a physical abrading of the stain to effect stain removal. Harsher abrasives, also known as polishing agents, than those used in typical non-whitening toothpaste preparations, are employed in this approach. Most, if not all of these preparations are toothpastes, gels or powder dentifrices. The mechanical process may be supplemented or even replaced by a chemical process which may involve an oxidative or enzymatic step to effect stain removal.

One such chemical process utilizes a tooth whitening or bleaching formulation applied to a stained tooth surface for a specified period, after which the formulation is removed. Oxidizing agents represent one of the most widely distributed and utilized class of active agents in commercially available tooth whitening or bleaching products. Peroxide-containing agents such as carbamide peroxide, hydrogen peroxide and calcium peroxide are the most commonly used oxidizing agents. Typically these products need to remain in the mouth for a period of time often greater than 60 minutes per day and may lose their whitening efficacy with time. Prolonged exposure of the teeth to these whitening compositions may have numerous adverse effects on the teeth, including tooth sensitivity. In order to address the problems associated with peroxide-containing products, the applicants have considered alternative non-peroxide tooth whitening compositions, eg compositions that generate chlorine dioxide, ie chlorine dioxide compositions. Chlorine dioxide is a known whitening or bleaching material and products, for example Samtweiss Beauty Kur, sold in Germany, Austria and Switzerland under the Odol Med 3 brand name, use chlorine dioxide as the bleaching agent. With all commercially available chlorine dioxide whitening products the chlorine dioxide is generated in-situ by the acidification of an aqueous chlorite source. The chlorite source may be an alkali metal chlorite or chlorate with alkali metal chlorites being preferred, for example sodium chlorite. Since chlorine dioxide is not stable for extended periods of time it is necessary to prepare, package and store two separately formulated components up until the point of use.

WO 98/04235 (Montgomery) describes a two component tooth whitening composition comprising chlorine dioxide prepared by admixture of sodium chlorite and a water soluble acid material known as an acidulant. The acidulant may be selected from the group including citric acid, malic acid, fumaric acid and other non-toxic, orally acceptable acidulants. Both the acidulant and the resulting mixture preferably have a pH in range of from about 3.0 to 4.5. U.S. Pat. No. 5,944,528 (Montgomery) similarly describes the preparation of a composition in which the acidulant in the above reaction is a gel with a pH of less than 6.0, preferably in the range of from about 3.0 to 4.5.

The reaction of sodium chlorite with an acidulant as described above is slow. This will, when used in the preparation of a tooth whitening composition, limit the yield of chlorine dioxide which in turn will limit the ability of the composition to bleach and whiten the teeth. Furthermore it is necessary to maintain a low pH, that is, in the range of 3.0 to 4.5 throughout the reaction in order to generate any significant quantity of chlorine dioxide. As the critical dissolution pH of enamel is approximately 5.5, maintaining such a low pH for any period of time is not advisable as enamel from the teeth may become eroded causing damage and leaving them in a highly sensitive state which may require further dental intervention. It has now become desirable to provide a composition capable of generating chlorine dioxide that can whiten the teeth in a relatively short period of time whilst at the same time avoiding the problems associated with chlorine dioxide products prepared with acidulants.

It is an object of the present invention to provide an effective chlorine dioxide tooth whitening composition that when prepared and is ready to apply to the tooth has a pH value of 6.0 or above and does not require the use of an acidulant to generate the chlorine dioxide. This object is met according to the invention by the provision of a tooth whitening composition comprising chlorine dioxide that is prepared by reacting a chlorite material with an organic acid anhydride.

In accordance with the present invention there is provided a tooth whitening composition comprising a chlorine dioxide precursor, an organic acid anhydride and an orally acceptable carrier,

The term “precursor” means an intermediate compound that when activated will convert to a specific functional material. In the present case the functional material is chlorine dioxide. The chlorine dioxide precursor will be selected from chlorite salts, eg alkali metal chlorites, such as potassium chlorite and sodium chlorite, alkaline earth chlorites, such as calcium chlorite, transition metal salts and organic salts and materials where the chlorite has been ionically adsorbed to a surface (eg ionic exchange resins, zeolites, silicas). A specific embodiment of the invention includes a precursor which is an alkali metal chlorite, such as sodium chlorite are preferred. Sodium chlorite is available commercially as a 3.35% solution from Bio-Cide International, 2845 Broce Drive, Norman, Okla. 73072, USA.

Any orally acceptable carrier may be used although one generally used in conventional oral compositions will be preferred. The orally acceptable carrier will aid the mixing of the two components as well as giving substantivity to the tooth surface during bleaching. Orally acceptable carriers may include water, surfactant, thickening agent and humectant as well as other optional extras normally included in an orally acceptable formulation.

The organic acid anhydride will activate the chlorine dioxide precursor on contact, eg. when physically mixed together. The organic acid anhydride may be selected from maleic anhydride, acetic anhydride, phthalic anhydride, octadecenyl succinic anhydride and polymeric anhydrides eg copolymers of methyl vinyl ether and maleic anhydride (eg. Gantrez AN). Suitably the organic acid anhydride will be succinic anhydride.

Succinic anhydride is available commercially from Shaanxi Rejoy Fine Chemical Co Ltd, 151 Hancheng Rd, Xian, China.

It should be noted that the by-products of the succinic anhydride/sodium chlorite reaction may be acidic and therefore a buffer, for example disodium succinate may be added to the composition to avoid the pH dropping to levels that may be considered erosive to tooth enamel.

Suitably the total amount of chlorine dioxide precursor present in the whitening composition of the invention is in the range of from 0.001 to 5% w/w, more suitably in the range 0.01 to 3% w/w, and most suitably in the range 0.05 to 2.5% w/w.

Suitably the total amount of organic acid anhydride present in the whitening composition is in the range of from 0.01 to 15.0% w/w, more suitably in the range 0.05 to 5.0% w/w.

In order to prevent the sodium chlorite reacting with the anhydride it will be necessary, prior to its intended use, to keep the two components physically separated from each other The composition may be prepared, packaged and stored as either two separate phases each containing one of the above components or as one phase where the components are for example encapsulated, or in a two layer strip or in a composition with a solvent where the reaction between the components is not initiated by the solvent.

Depending on the mode of application, a variety of auxiliary materials may be added to either phase to for example thicken the compositions or to improve both its performance and aesthetic qualities.

Suitable thickeners include alkyl vinyl ether/maleic anhydride copolymer, alkyl vinyl ether/maleic acid copolymer, alkali metal or an amine salt of alkyl vinyl ether/maleic acid copolymer, partially or fully crosslinked alkyl vinyl ether/maleic anhydride copolymer, vinyl acetate copolymer, polyacrylates, polyurethane interpolymers, chitosan, poly(acrylic acid), poly(vinyl alcohol), poly(vinyl alcohol-g-ethylene glycol) copolymer, cellulose derivatives, hydroxy-propyl-methyl cellulose, hydroxyl-ethyl cellulose, hydroxy-propyl cellulose, poly(ethylene oxide), polypropylene oxide), Polyquaterium-11, Polyquaterium-39, poloxamer, carbomer, gelatin, starch, alginic acid, salt of alginic acid, natural gums such as gum karaya, xanthan gum, guar gum, arabic gum tragacanth, or any combinations thereof.

The whitening composition may also contain ingredients that further enhance benefits to the oral cavity and teeth. Such ingredients may also be called additives and include for example: an antimicrobial agent, a mineralization compound, a stain prevention compound, a desensitization compound, an anti-calculus agent, a flavoring agent, a colouring agent, a sweetening agent, an anti-inflammatory agent, an antioxidant, a volatile sulfur scavenger, an odorant neutralizer, and/or a vitamin, a humectant or a pH adjuster. The whitening composition may also contain a penetration enhancer, a plasticizer, a preservative, a surfactant or wetting agent, an anesthetic, an anti-allergenic, a pharmaceutical, or any combinations thereof.

The following antimicrobial agents may be used in the composition: polyphenols (e.g. triclosan) zinc salts, stannous fluoride, chlorhexidine, hexetidine, sanguinarine, benzalkonium chloride, salicylanilide, domiphen bromide, cetylpyridinium chloride (CPC), tetradecylpyridinium chloride (TPC), N-tetradecyl-4-ethylpyridinium chloride (TDEPC), octenidine, delmopinol, octapinol, and other piperidine derivatives, nicin preparations, zinc/stannous ion agents, antibiotics such as amoxicillin, tetracycline, doxycycline, minocycline, and metronidazole, and analogs and salts of the above, essential oils including thymol, menthol, eugenol, geraniol, carvacrol, citral, hinokitiol, eucalyptol, catechol, methyl salicylate, hydrogen peroxide, metal salts of chlorite, or any combinations of all of the above.

Mineralising compounds may also be used in the present invention: sodium monoflurophosphate, potassium monofluorophosphate, magnesium monofluorophosphate, acidulated fluorophosphate, amine fluoride, water-soluble salts of fluoride, such as, sodium fluoride, potassium fluoride, calcium fluoride, stannous fluoride, sodium fluorosilicate, bis-salicylato-bis-fluorotitanium (IV), ammonium fluorosilicate, calcium salt, phosphate salt, calcium salt/phosphate salt, calcium salt/ionic fluoride sources, zinc salt/phosphate salt, or any combinations thereof.

Desensitising compounds may also be used in the present invention: water-soluble potassium salt including potassium nitrate, potassium citrate, potassium chloride, potassium bicarbonate, potassium oxalate, and tubular occlusion compounds (e.g., ferric oxalate), or any combinations thereof.

The following anti-calculus agents may be used in the invention: phosphates, pyrophosphates, polyphosphates, phosphonates (e.g. ethane-1-hydroxy-1,1-diphosphonate, 1-azacycloheptane-1,1-diphosphonate) polyphosphonates, polyacrylates and other polycarboxylates, ethylenediaminetetraacetic acid and other calcium chelators, carboxylic acids and their salts, zinc salts (e.g. sodium zinc citrate),

PVM/MA copolymer or other polymers which interfere with crystal nucleation or growth, or any combinations thereof.

The following anti-inflammatory agents may also be used in the present invention: non-steroidal anti-inflammatory agents, such as, ketorolac, flurbiprofen, ibuprofen, naproxen, indomethacin, aspirin, ketoprofen, piroxicam, meclofenamic acid, or any combinations thereof. Also, steroidal and non-steroidal anti-inflammatory agents and plant extracts that have demonstrated anti-inflammatory activities may be used.

The following antioxidants may be used in the present invention: Vitamin E, ascorbic acid, uric acid, kojic acid, coenzyme compounds (e.g. coenzyme Q-10), carotenoids, Vitamin A, flavonoids and polyphenols, herbal antioxidants, melatonin, aminoindoles, lipoic acids, or any combinations thereof.

Other suitable antioxidants include: rosemary extract, tocopherol, a derivative of tocopherol including a tocotriene, carotene, a carotenoid, a phenolic antioxidant including a phenolic acid, a bioflavonoid, a plant extract, curcumin, tetrahydrocurcumin, camphorol, quercetine, epigenine, or any mixtures thereof.

The following vitamins may be used in the present whitening formulation: Vitamin K, retinol (Vitamin A), tocopherol (Vitamin E), ascorbic acid (Vitamin C) or any combinations thereof.

The following flavouring agents may be used in the present invention: flavoring oils, e.g., oils of spearmint, peppermint, wintergreen, sassafras, clove, sage, eucalyptus, marjoram, cinnamon, lemon, menthol, anethole, thymol, parsley oil, oxanone and orange, alpha-irisone, cassia, marjoram, oils thereof, propenyl guaethol, and methyl salicylate. Sweetening agents including, but not limited to, sucrose, lactose, maltose, sorbitol, xylitol, sodium cyclamate, sucralose, acesulfame-K, aspartame, and sodium saccharin. Any combinations of the preceding flavoring agents are also suitable for use in the formulation.

The additives used in any given composition will be compatible both with each other and with the essential ingredients of the composition such that there is no interaction which would impair the performance of the ingredients. All additives must of course be non-toxic and of sufficient purity to render them suitable for human use.

According to one embodiment of the invention, to whiten the teeth, two phases comprising sodium chlorite and succinic anhydride respectively are mixed thoroughly prior to placing the entire composition into a custom made dental tray. The dental tray is then placed in the mouth for a predetermined period of time. After a predetermined period of time the tray is removed from the mouth and any excess mixed gel remaining on the teeth may be removed from the user's teeth by any convenient means e.g. brushing and/or by rinsing with a mouthwash.

According to a further embodiment of the present invention, compositions according to the present invention may be applied topically to the teeth as appropriate in the form of lotions (eg. mouthrinses), gels, mousses, sprays or aerosols. For example, the composition may be painted onto the teeth. The term “painted onto the teeth” above is intended to encompass all manner of applying the whitening composition to teeth and includes sponging, coating, daubing, spraying, wiping and rubbing.

In a further embodiment the whitening composition may be deposited on the surface of, or incorporated in, a dissolvable or non dissolvable strip for application to the tooth surface. The strip can either be in the form of a single or multilayer system.

According to the present invention there is also provided a method of whitening teeth comprising:

a. taking a first phase comprising a chlorine dioxide precursor;

b. taking a second phase comprising an organic acid anhydride;

c. thoroughly mixing said first and second phases and filling a suitable mouth tray;

d. applying the mixture to and maintaining it in contact with the teeth for a plurality of minutes per day;

e. repeating steps c and d for multiple days e.g. up to fourteen days, to thereby whiten the teeth.

According to a further embodiment of the present invention there is also provided a method of whitening teeth comprising:

a. preparing a first phase comprising a chlorine dioxide precursor;

b. preparing a second phase comprising an organic acid anhydride;

c. thoroughly mixing said first and second phases and filling a suitable mouth tray;

d. applying the mixture to and maintaining it in contact with the teeth for a plurality of minutes per day;

e. repeating steps c and d for multiple days e.g. up to fourteen days, to thereby whiten the teeth.

The present invention is illustrated by the following examples but is not limited thereby.

### 1. Sodium Chlorite Aqueous Gel

| Ingredient | % w/w |
|---|---|
| 10.3% sodium chlorite solution* | 0.95 |
| Hydroxypropyl cellulose | 2.00 |
| Hydroxyethyl cellulose | 2.00 |
| Disodium succinate | 2.00 |
| Succinic anhydride | 0.20 |
| Deionised Water | to 100.00 |

*available from Bio-Cide International

The sodium chlorite solution, disodium succinate and water were mixed until dissolved. The hydroxypropyl cellulose and hydroxyethyl cellulose were added with stirring until a homogenous gel was obtained. The succinic anhydride was added to the gel immediately prior to use and stirred again until homogenous. The resulting gel was then applied to either a tray or strip for immediate use.

### 2. Sodium Chlorite Aqueous Gel

| Ingredient | % w/w |
|---|---|
| 10.3% sodium chlorite solution* | 0.49 |
| Hydroxypropyl cellulose | 2.00 |
| Hydroxyethyl cellulose | 2.00 |
| Disodium succinate | 2.00 |
| Succinic anhydride | 0.20 |
| Deionised Water | to 100.00 |

*available from Bio-Cide International

The sodium chlorite solution, disodium succinate and water were mixed until dissolved. The hydroxypropyl cellulose and hydroxyethyl cellulose were added with stirring until a homogenous gel was obtained. The succinic anhydride was added to the gel immediately prior to use and stirred again until homogenous. The resulting gel was then applied to either a tray or strip for immediate use.

### 3. Sodium Chlorite Dry Powder For Reconstitution

| Ingredient | % w/w |
|---|---|
| Xanthan gum | 22.00 |
| Pre-neutralised Noveon AA1 | 53.00 |
| Polypore (containing sodium chlorite) | 13.00 |
| Polypore (containing succinic anhydride) | 12.00 |

7g of Noveon AA-1 was dispersed in 300 g of water and 23 g of 18% NaOH added to neutralise whilst stirring. The gel was then dried at 105° C. and the resulting dry solid was micronised to form the pre-neutralised material. All of the dry solid ingredients were then mixed thoroughly and stored in a sealed jar. The powder can be incorporated in a tray or strip so that when wetted forms a chlorine dioxide containing, substantive gel.

Noveon AA1 is a cellulose derivative and is available from Noveon Inc, 9911 Brecksville Rd, Cleveland, Ohio, USA.

Polypore is an encapsulating material available from Amcol Health & Beauty Solutions, Inc.

### 4. Sodium Chlorite Wax

| Ingredient | % w/w |
|---|---|
| Antaron V-220 | 92.00 |
| Polypore (containing sodium chlorite) | 4.00 |
| Polypore (containing succinic anhydride) | 4.00 |

The Antaron was melted and then cooled to 40° C. The polypore materials were added with stirring. Once cooled to room temperature the brittle solid was ground to form a powder that liberates chlorine dioxide once wetted with water. The powder was then incorporated into a tray or strip.

Antaron V-220 is a wax and is available from ISP, 1361 Alps Rd, Wayne, N.J., USA. Polypore is an encapsulating material available from Amcol Health & Beauty Solutions, Inc.

### 5. Sodium Chlorite Mouthrinse

Two chlorine dioxide-containing mouthrinse (MR) compositions were prepared as per the table below.

| | % w/w | % w/w |
|---|---|---|
| Ingredient | MR1 | MR2 |
| 10.3% NaClO₂ Solution | 2 | 5 |
| Disodium succinate | 2 | 2 |
| Succinic Anhydride | 0.26 | 0.34 |
| Deionised Water | to 100 | to 100 |

The succinic anhydride was added only immediately prior to use.

### Data

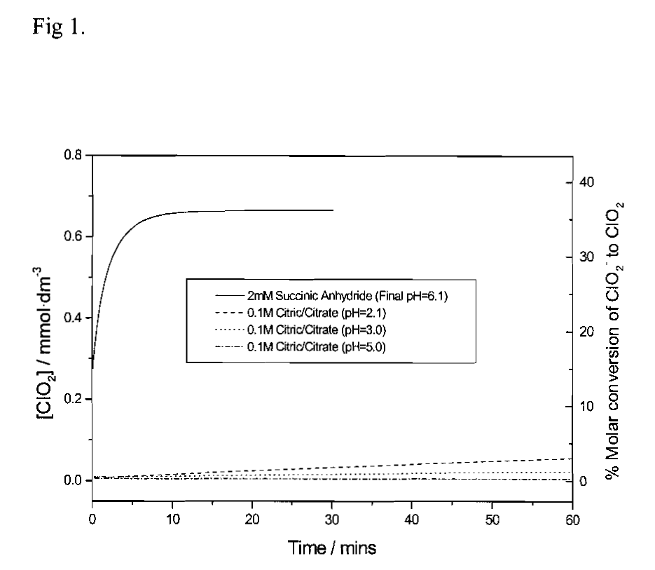

FIG. 1 shows the generation of chlorine dioxide from sodium chlorite at room temperature as a function of time. 1.8 mmol·dm⁻³ of sodium chlorite in aqueous solution was acidified with 0.1 mol·dm⁻³ citric acid/sodium citrate buffer at various pH's. The amount of chlorine dioxide produced was measured spectrophotmetrically and the concentration calculated using an extinction coefficient of 1250M⁻¹ cm⁻¹ at 359 nm. FIG. 1 shows that practically no chlorine dioxide is formed at pH 5.0, whilst even at pH 2.1 only approximately 4% of the chlorite has been converted to chlorine dioxide after one hour. In contrast to this FIG. 1 also shows the effect of adding 2 mmol·dm⁻³ of succinic anhydride and 20 mmol·dm⁻³ of disodium succinate to a 1.8 mmol·dm⁻³ aqueous solution of sodium chlorite, in this case no citric/citrate buffer was added and the final pH after mixing was 6.1. The succinic anhydride has converted 35% of the chlorite to chlorine dioxide in a period of 15 minutes, a far greater and more rapid turnover than generated by the acid reaction. It should be noted that the by-products of the succinic anhydride reaction appear to be acidic and require the presence of a buffer (disodium succinate in the example above) to avoid the pH dropping to levels that may be considered erosive to enamel.

### Whitening Data 1

### Method

Bovine teeth were used and L* (from CIE 1976 L*a*b* colour space scale) was measured at the start of the experiment using a spectrocolorimeter. Example 2 was applied to the teeth and these were placed into a container such that a liquid substantivity challenge was applied to the treated teeth. At the end of the treatment time the teeth were rinsed and dried and L* was remeasured using a spectrocolorimeter. This method was repeated for a number of treatments to mimic multiple use of the product. At the end of the experiment the overall change in L* (i.e ΔL*) was calculated.

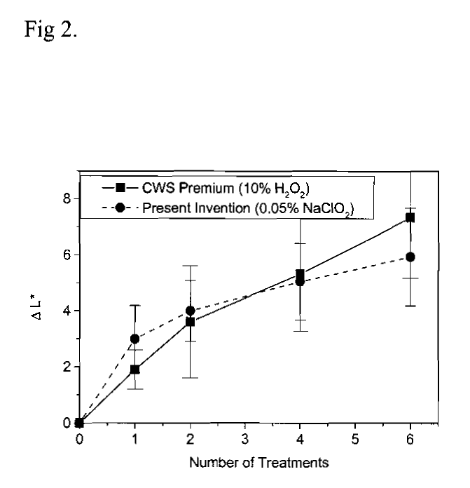

As shown in FIG. 2, a composition of the present invention initially containing 0.05% sodium chlorite performs as well as Crest White Strips (CWS) Premium (containing 10% H₂O₂) in a bovine-bleaching assay.

### Whitening Data 2

### Method

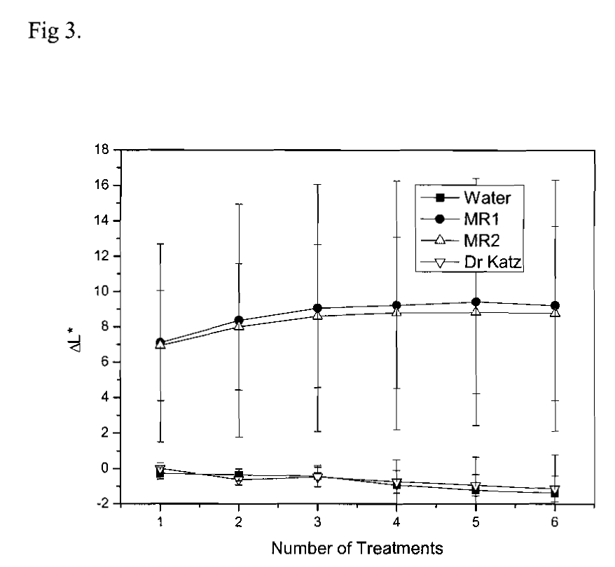

Bovine teeth were used and L* (from CIE 1976 L*a*b* colour space scale) was measured at the start of the experiment using a spectrocolorimeter. The teeth were soaked for 2 minutes in either MR1, MR2 of Example 5 above or water or Dr Katz Therabrite+ mouthrinse prepared as per manufacturers instructions (contains stabilized oxychlor compounds). At the end of the treatment time the teeth were rinsed, dried and L* was remeasured using a spectrocolorimeter. This method was repeated for 6 treatments to mimic multiple use of the product. At the end of the experiment the overall change in L* (i.e ΔL*) was calculated. FIG. 3 shows that the water and the commercial Dr Katz mouthrinse did not whiten teeth, despite the Dr Katz mouthrinse containing stabilised oxychlor compounds. However the 2 formulations MR1 and MR2 whitened teeth highly effectively.

1. A tooth whitening composition comprising a chlorine dioxide precursor, an organic acid anhydride and an orally acceptable carrier.

2. A tooth whitening composition according to claim 1 wherein the chlorine dioxide precursor is an alkali metal chlorite.

3. A tooth whitening composition according to claim 2 wherein the alkali metal chlorite is sodium chlorite.

4. A tooth whitening composition according to claim 1 wherein the organic acid anhydride is succinate anhydride.

5. A tooth whitening composition according to claim 1 wherein the orally acceptable carrier comprises a thickener material.

6. A tooth whitening composition according to claim 1 additionally comprising a buffering material.

7. A tooth whitening composition according to claim 1 in the form of a lotion, gel, mousse, spray or aerosol.

8. A tooth whitening composition according to claim 1 in the form of a mouthrinse.

9. A kit of parts comprising a tooth whitening composition according to claim 1, a container for housing the composition and an applicator for applying the composition to teeth to be whitened.

10. A kit of parts according to claim 9 wherein the applicator is a mouth tray.

11. A method of whitening teeth comprising:
a. taking a first phase comprising a chlorine dioxide precursor;
b. taking a second phase comprising an organic acid anhydride;
c. thoroughly mixing said first and second phases and filling a suitable mouth tray;
d. applying the mixture to and maintaining it in contact with the teeth for a plurality of minutes per day;
e. repeating steps c and d for multiple days e.g. up to fourteen days, to thereby whiten the teeth.

12. (canceled)
